(19) United States
(12) Patent Application Publication
(10) Pub. No.: US 2008/0208142 A1
(43) Pub. Date: Aug. 28, 2008

(54) Dose Setting Mechanism for an Injection Device

(75) Inventor: Claus Schmidt Moller, Fredensborg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, BAGSVAERD (DK)

(21) Appl. No.: 11/816,917

(22) PCT Filed: Feb. 24, 2006
(86) PCT No.: PCT/EP2006/001708

§ 371 Date: Aug. 23, 2007

(30) Foreign Application Priority Data
Feb. 28, 2005 (EP) 05075476.1

(51) Int. Cl. A61M 5/315

(52) U.S. Cl. 604/208

(57) ABSTRACT

A dose setting mechanism for an injection device, e.g. for repetitive self-injection of e.g. insulin or growth hormone. Has a dose setting member, a driving element for causing a set dose to be injected, and a locking member being connected to the driving element. The position of the locking member is indicative of the remaining amount of liquid in a cartridge. When the amount is below a critical lower value, the locking member assumes a locking position where it prevents the dose setting member from being operated to set a dose.

Ensures that a dose can only be set if the remaining amount of liquid in the cartridge is sufficient to ensure proper mixing of the contents of the liquid. At the same time ensures optimal usage of the available liquid. Particularly suitable for injection devices for long acting or mix insulin.

### FIELD OF THE INVENTION

The present invention relates to a dose setting mechanism for an injection device, preferably of a kind being suitable for repetitive self-injections of medication, such as insulin or growth hormone. The dose setting mechanism has a locking feature which prevents the setting of a dose if a remaining amount of liquid in a cartridge of the injection device is below a predefined critical lower value. Furthermore, the present invention relates to an injection device comprising the dose setting mechanism.

### BACKGROUND OF THE INVENTION

Over time the active elements of some medications precipitate. As an example, this is the case for long acting and mix insulin types. Thus, in order to ensure an even concentration of the active elements in injected medication, the cartridge or the injection device which contains the medication must be shaken prior to injection. However, the smaller the amount of medication to be shaken is, the harder it is to ensure an even concentration by shaking. Accordingly, it is desirable to provide an injection device which prevents a user from setting a dose, and thereby from injecting medication, when the amount of the medication is below a predetermined level. At the same time, if the remaining amount is above this level, it must be possible to inject the entire remaining amount of the medication.

Mechanisms for preventing setting of a dose which is larger than the amount of liquid remaining in the cartridge are known. Thus, US 2004/0068236 A1 discloses an end-of-content arrangement for preventing a dose setting member of an injection device to be set to a dose larger than the medicament remaining in the injection device. The arrangement comprises an indentation provided on the housing or on the dose setting and injection member which indentation arrest a stop member when less than one dose unit is left in the cartridge. The stop member is coupled to the dose setting member such that the interaction between the indentation and the stop member prevents a dose larger than the remaining medication in being set. The arrangement of US 2004/0068236 A1 prevents setting of a dose when only one dose unit is left in the cartridge. Accordingly, the cartridge will always be discarded with one dose unit still left in it. One dose unit is not sufficient to ensure proper mixing of the medication. Therefore, in case the arrangement described above should be used for this purpose, the limit should be chosen somewhat higher, e.g. at 12 dose units. Accordingly, the cartridge would always be discarded with 12 dose units of medication still remaining therein, and the medication of the cartridge would therefore not be used to the maximum possible extent. This is a disadvantage.

US 2004/0097883 A1, WO 2004/028598 A1 and US 2004/0236285 A1 all disclose injection devices in which setting of a dose larger than the remaining amount of medication in the cartridge is prevented. However, setting of a dose corresponding to the remaining amount of medication is always possible, and the problem outlined above with respect to proper mixing of the drug is therefore not solved by these devices.

### SUMMARY OF THE INVENTION

It is, thus, an object of the present invention to provide a dose setting mechanism for an injection device, the dose setting mechanism being adapted to ensure proper mixing of the medication to be injected.

It is a further object of the present invention to provide a dose setting mechanism for an injection device, where the dose setting mechanism can only be operated if a sufficient amount of medication remains to ensure a proper mixing of the medication.

It is an even further object of the present invention to provide a dose setting mechanism which allows an optimal usage of liquid available in a cartridge while ensuring a proper mixing of the liquid.

It is an even further object of the present invention to provide an injection device being adapted to ensure proper mixing of medication to be injected.

According to a first aspect of the present invention the above and other objects are fulfilled by providing a dose setting mechanism for an injection device, the dose setting mechanism comprising:
a dose setting member being operable by a user to set a desired dose,
a driving element being adapted to drive a piston rod, thereby causing a set dose to be injected by the injection device,
a locking member being operatively connected to the driving element in such a way that movement of the driving element during injection of a set dose causes a movement of the locking member, the position of the locking member thereby being indicative of a remaining amount of liquid in a cartridge of the injection device, the locking member further being adapted to assume a locking position when the remaining amount of liquid is below a predefined critical lower value,
   wherein the locking member, when in the locking position, prevents the dose setting member from being operated to set a dose, and wherein the dose setting mechanism may be operated to set and inject a dose when the locking member is not in the locking position, even if injection of the set dose results in the remaining amount of liquid falling below the predefined critical lower value.

The injection device is preferably of the kind which is suitable for repetitive self-injection of medication, such as insulin or growth hormone. The injection device may be of the pen-shaped type, or it may have any other suitable shape.

In the present context the term ‘dose setting mechanism’ should be interpreted to mean the part(s) of the injection device being responsible for the setting of a desired dose. The dose setting mechanism thus comprises a dose setting member which the user operates in order to set a dose, as well as any internal parts being necessary in order to ensure that when a dose is subsequently injected, the injected dose is identical to the dose which the user believes he/she has set. Such parts may, e.g., comprise a scale drum, a ratchet, a piston rod, and/or any other suitable parts which are normally used when setting a dose in an injection device.

The dose setting member may be rotatable relatively to a housing of the injection device, e.g. in the form of a dose knob. In this case the user rotates the dose setting member in order to set a desired dose. Alternatively or additionally, the dose setting member may be operated by means of a translational movement, e.g. by pulling the dose setting member in a substantially axial direction or by rotating the dose setting member along a spiral path.

The locking member is operatively connected to the driving element in such a way that movement of the driving element during injection of a set dose causes a movement of the locking member. This movement of the locking member may, e.g., occur as the result of a relative movement between the driving element and a scale drum being operatively connected to the dose setting member. The locking member may be or form part of the driving element. Alternatively, it may be a separate part which is connected to the driving element in such a way that it is moved when the driving element is moved during injection of a set dose. The movement of the locking member may be identical to the movement of the driving element, i.e. in case the driving element rotates through a specific angle, the locking member is also caused to rotate through an identical angle, and/or in case the driving element is translated through a specific distance in a specific direction, the locking member is also caused to perform an identical translational movement. Alternatively, the movement of the locking member may be different from the movement performed by the driving element. Thus, a rotational movement of the driving element may cause the locking member to be translated and vice versa, or a rotation of the driving element through a specific angle may cause the locking member to be rotated through a smaller or larger angle, etc. What is important is that a movement of the driving element causes a movement of the locking member, and that the position of the locking member is therefore linked to how much liquid has been injected by the injection device since the current cartridge was inserted. Thereby the position of the locking device is indicative of the remaining amount of liquid in the cartridge.

The predefined critical lower value of the remaining amount of liquid defines a limit in the sense that when a user wishes to set a dose, and the remaining amount of liquid is above the predefined critical lower value it is safe to set a desired dose, and this should therefore be possible. If, on the other hand, the remaining amount of liquid is below the predefined critical lower value, injecting a dose of the remaining contents is not safe, and setting of a dose should accordingly not be possible. Preferably, the predefined critical lower value is chosen in such a way that in case the remaining amount of liquid is above the value, the amount is sufficient to ensure a proper mixing of the contents of the liquid, and in case the remaining amount of liquid is below the value, a proper mixing of the contents can not be ensured, and it is therefore not safe to set a dose. In case the medication is long acting or mix insulin, it will often not be possible to ensure proper mixing of the contents if the remaining amount of liquid in the cartridge is less than 12 international units (IU), and this could therefore advantageously be chosen as the predefined critical lower value. However, the predefined critical lower value may alternatively be chosen in accordance with other factors making it safe to set a dose when the remaining amount of liquid is above the value and unsafe to set a dose when the remaining amount of liquid is below the value.

When the locking member is in the locking position it prevents the dose setting member from being operated to set a dose, i.e. the locking member locks the dose setting member and/or one or more other parts which cooperate(s) with the dose setting member to set a dose. When the locking member is not in the locking position it is, on the other hand, possible to set and inject a dose up to the entire remaining amount of liquid in the cartridge, even if injection of the set dose results in the remaining amount of liquid falling below the predefined critical lower value. Thus, in case the remaining amount of liquid in the cartridge falls below the predefined critical lower value during injection of a set dose, the remaining part of the set dose will be injected regardless of the fact that the locking member assumes the locking position during the injection. Thereby it is ensured that a set dose is in fact injected, thereby allowing the user to keep track of the dose which has actually been injected. However, in this case setting of a new dose will be prevented by the locking member, because it is now in the locking position.

Thus, optimal usage of the available contents of liquid in the cartridge is provided, and at the same time it is ensured that a proper mixing of the contents is possible when a dose is set, i.e. it is ensured that it is safe to inject a set dose, or, in other words, setting of a dose is prevented if it is not safe to inject a set dose.

The locking member may be adapted to move along a longitudinal axis of the injection device during injection of a set dose, i.e. it may be adapted to perform a translational movement. This is particularly advantageous if the injection device has an elongated shape, in which case the longitudinal axis is preferably defined by the elongated shape. Alternatively or additionally, the locking member may be adapted to perform a rotational movement during injection of a set dose. However, it should be ensured that the movement of the locking member during injection of a set dose does not prevent injection of the set dose. Thus, in case the locking position is defined by the locking member engaging with another part of the injection device, and in case the locking member rotates during injection of a set dose, this rotation may not be prevented or inhibited in case the locking member and the other part are moved into engagement during injection of a set dose. This may, e.g., be achieved by letting the other part perform a similar rotational movement during injection of a set dose.

In one embodiment the locking member may further be operatively connected to the dose setting member in such a way that movement of the dose setting member during dose setting causes a movement of the locking member. The movement of the locking member during dose setting may be different from the movement of the locking member during injection of a set dose. Thus, the movements may be in opposite directions, the movement during injection of a set dose may be purely translational while the movement during dose setting comprises a rotational component, or vice versa, etc. Such a difference in the movements will preferably ensure that injection of a set dose is not prevented in case the remaining amount of liquid falls below the predefined critical lower value during injection of the dose. In this embodiment, when the locking member is in the locking position, it is prevented from performing the movement which is caused by the movement of the dose setting member during setting of a dose. This, in turn, prevents the dose setting member from moving, whereby setting of a dose is prevented.

In a preferred embodiment the locking member may be adapted to perform a substantially translational movement along a longitudinal axis of the injection device during injection of a set dose, and adapted to perform a rotational movement about said longitudinal axis during setting of a dose. In this embodiment the locking member will preferably be prevented from rotating when it is in the locking position, but it will not be prevented from performing the substantially translational movement. Thereby injection of a set dose is allowed, but setting of a new dose is prevented, when the locking member is in the locking position.

In this case the locking member may comprise a set of teeth being adapted to engage one or more mating teeth in such a way that rotational movement of the locking member is prevented, the engagement of said teeth thereby defining the locking position of the locking member. Such a construction will not prevent a substantially translational movement of the locking member. In a very simple construction of this type the locking member, and thereby the set of teeth, is moved along with the driving element and eventually into engagement with the mating teeth which are typically fixed relatively to a housing of the injection device. Alternatively, the locking member may be moved in a substantially opposite direction as compared to the direction of movement of the driving element.

In some injection devices the driving element is coupled to a scale drum during setting of a dose. Thereby the driving element performs a spiral movement along with the scale drum during setting of a dose. However, during injection of a set dose the dose setting member and/or the driving element is/are typically coupled to a housing of the injection device. This prevents the driving element from rotating during injection of a set dose, and it therefore performs an axial movement. When a set dose has been injected, the dose setting member and/or the driving element is normally decoupled from the housing in order to allow it to rotate along with the scale drum during setting of a new dose.

In one embodiment of the invention the locking position of the locking member may be a position in which the locking member prevents a decoupling of the dose setting member and/or the driving element from a housing of the injection device. Thus, in this embodiment, when the remaining amount of liquid in the cartridge falls below the predefined critical lower value, and the locking member therefore assumes the locking position, the locking member will be in a position, where it prevents the decoupling described above of the dose setting member and/or the driving element from the housing. Thereby the driving element will be prevented from rotating along with the scale drum, and setting of a dose is therefore prevented. This may, e.g., be achieved in the following manner. During injection of a dose the locking member is moved in a direction away from an end of the injection device normally holding a needle, i.e. the locking member is moved towards an end of the injection device where the dose setting member is normally positioned. The locking member is moved via a threaded portion on the driving element. When the remaining amount of liquid in the cartridge falls below the predefined critical lower value, the locking member will be positioned in a distance from a flange on the scale drum which is smaller than the distance needed to allow the decoupling described above. When a new dose is to be set, the locking member will accordingly abut the flange, thereby preventing the decoupling.

According to a second aspect of the present invention the above and other objects are fulfilled by providing an injection device comprising a dose setting mechanism according to the first aspect of the present invention. The injection device is preferably of the kind which is suitable for repetitive self-injections of medication, such as insulin or growth hormone. The injection device preferably has an elongated shape, such as a pen-like injection device.

### BRIEF DESCRIPTION OF THE DRAWINGS

The invention will now be further described with reference to the accompanying drawings, in which:

FIG. 1 is a cross sectional view of an injection device according to an embodiment of the invention with a locking member in a start position,

FIG. 2 is a cross sectional view of the injection device of FIG. 1 with the locking member in a position immediately before assuming a locking position,

FIG. 3 is a cross sectional view of the injection device of FIG. 1 with the locking member in the locked position, and

FIG. 4 is an exploded view with parts broken away of the injection device of FIG. 1.

### DETAILED DESCRIPTION OF THE DRAWINGS

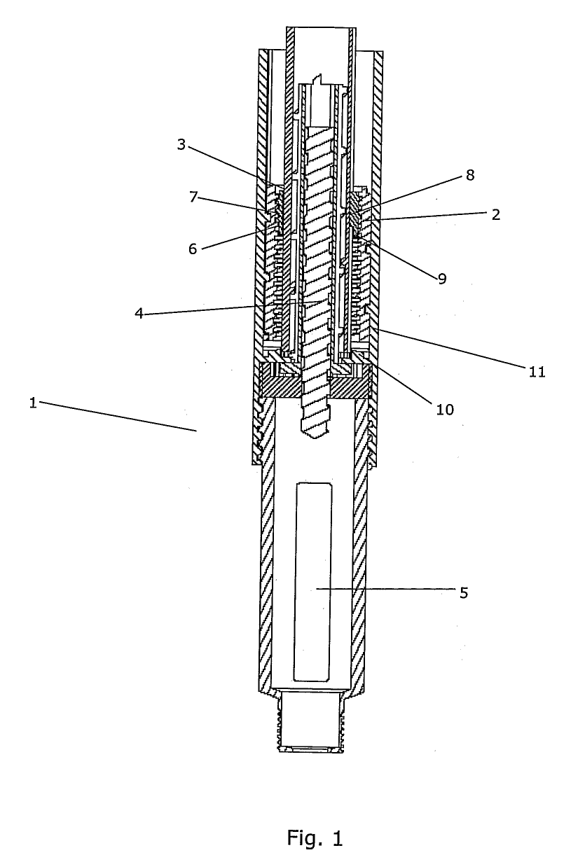

FIG. 1 is a cross sectional view of an injection device 1 according to an embodiment of the invention. The injection device 1 comprises a scale drum 2 which is connected to a dose setting member (not shown) and being adapted to be moved along with the dose setting member during setting of a dose. Thus, during setting of a dose the scale drum 2 performs an outward spiral movement, i.e. it rotates and moves axially in a direction away from the remaining parts of the injection device 1. During injection of a set dose the scale drum 2 performs an opposite movement, i.e. it rotates and translates back towards the remaining parts of the injection device 1. The scale drum 2 is adapted to display the set dose to a user.

The injection device further comprises a driving element 3 being adapted to drive a piston rod 4 during injection of a dose, the piston rod 4 thereby causing a movement of a piston (not shown) in a cartridge 5, thereby causing a set dose to be injected. During setting of a dose the driving element 3 performs an outward spiral movement along with the scale drum 2. During injection of a set dose the driving element 3 is prevented from rotating and therefore performs a pure axial movement back towards the remaining parts of the injection device 1.

Between the scale drum 2 and the driving element 3 there is positioned a locking member 6. The locking member 6 is connected to the scale drum 2 via a thread connection 7 comprising an outer thread on the locking member 6 and an inner thread on the scale drum 2. The locking member 6 is also connected to the driving element 3 via a key/groove connection 8 comprising an inner key on the locking member 6 and a groove on the driving element 3. Thus, during setting of a dose the locking member 6 is rotated and translated along with the scale drum 2 and the driving element 3, i.e. the locking member 6 is not moved axially relatively to the scale drum 2 during setting of a dose. During injection of a set dose the locking member 6 is moved along with the driving element 3, i.e. it performs a pure axial movement back towards the remaining parts of the injection device 1. Thereby the locking member 6 is moved axially relatively to the scale drum 2 during injection of a dose, and the position of the locking member 6 thereby indicates the remaining amount of liquid in the cartridge 5.

The locking member 6 is provided with a set of teeth 9 being adapted to engage with a corresponding set of teeth 10 attached to a part of a housing 11 of the injection device 1. When the set of teeth 9 on the locking member 6 and the corresponding set of teeth 10 on the housing 11 engage, the locking member 6 will be prevented from rotating, and thereby it is not possible to set a dose. By positioning the locking member 6 appropriately the teeth 9, 10 will engage when the remaining amount of liquid in the cartridge 5 is below a lower limit, and this lower limit can be chosen in such a way that when a larger amount of liquid remains the medication can be properly mixed by shaking, and if a lower amount remains it is not safe to assume that the medication is properly mixed by shaking. Thereby it is ensured that a dose can not be set if the remaining amount of liquid is too small to ensure a proper mixing of the medication, but it will be possible to set a dose if it is safe to assume that the remaining amount of liquid is sufficient to ensure proper mixing, even if injecting the set dose means that the new remaining amount of liquid is well below the lower limit. However, in FIG. 1 the locking member 6 is in a position being as far away from the corresponding set of teeth 10 as possible. This indicates that the cartridge 5 has been inserted in the injection device 1 very recently, i.e. the locking member 6 is in a ‘start position’, and a desired dose can be set.

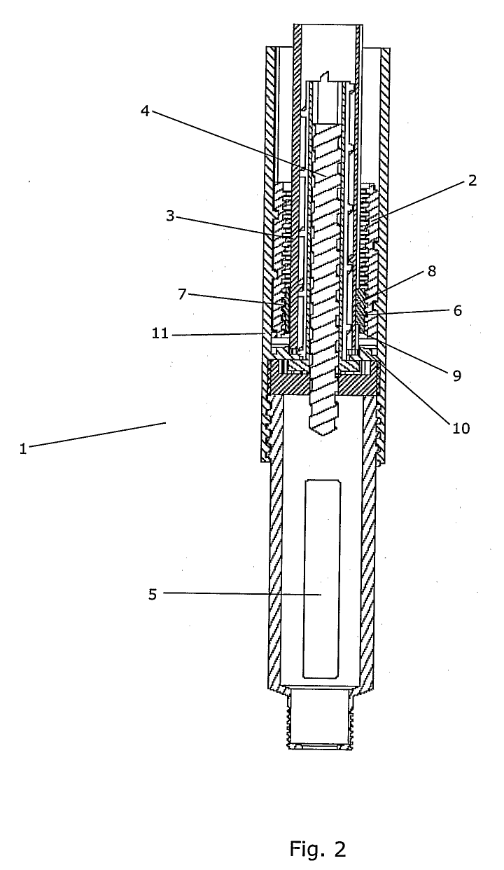

FIG. 2 is a cross sectional view of the injection device 1 of FIG. 1. However, in this case the locking member 6 is in a position immediately above the corresponding set of teeth 10. Since the teeth 9, 10 do not engage it is possible to rotate the locking member 6, and it is therefore possible to set a desired dose. Since the locking member 6 is moved away from the corresponding set of teeth 10 during setting of a dose, the teeth 9, 10 will not be moved into engagement during the dose setting, even though the locking member 6 is positioned very close to the corresponding set of teeth 10 before the dose setting is commenced. However, when the set dose is subsequently injected, the locking member 6 is moved axially relatively to the scale drum 2, and thereby the teeth 9, 10 will be moved into engagement. Because the locking member 6 performs a pure axial movement during injection of a set dose, the teeth 9, 10 will be moved into engagement, but injection of the set dose will not be prevented or inhibited. However, the mutual engagement of the teeth 9, 10, will prevent a new dose from being set because the locking member 6 will be prevented from rotating once the teeth 9, 10 engage.

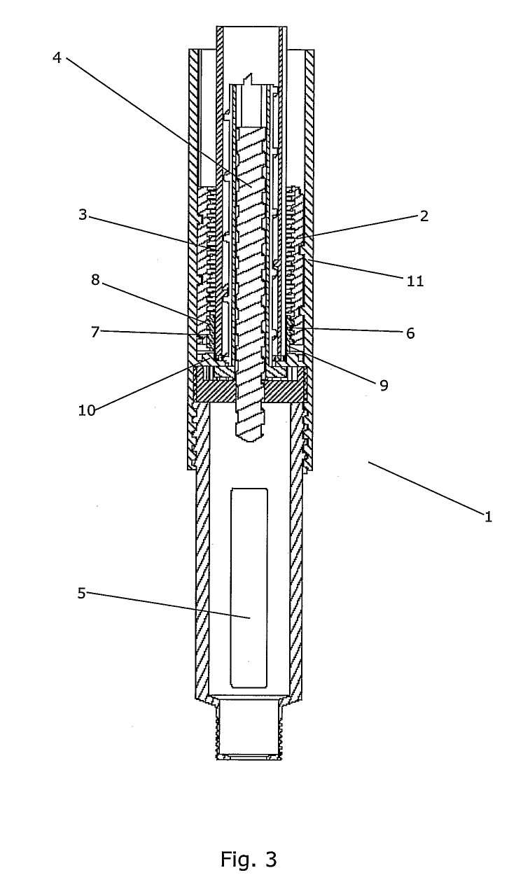

FIG. 3 shows this situation. The locking member 6 is in a position where the teeth 9, 10 engage, thereby preventing the locking member 6 from rotating, and a dose can therefore not be set. It is thereby prevented that a dose is set when the remaining amount of liquid in the cartridge 5 is below a predefined critical lower limit.

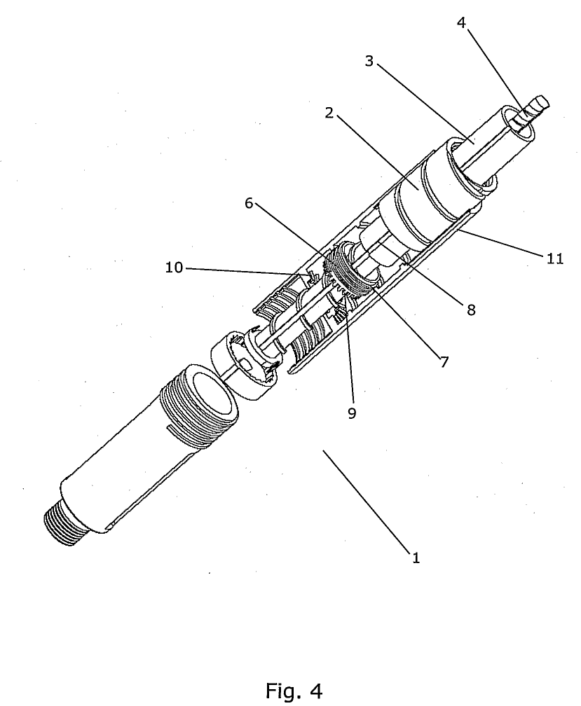

FIG. 4 is an exploded view of the injection device 1 of FIG. 1. Part of the housing 11 has been broken away for clarity. In FIG. 4 the shapes and mutual positions of the various parts can be readily seen.

1. A dose setting mechanism for an injection device, the dose setting mechanism comprising:
a dose setting member being operable by a user to set a desired dose,
a driving element being adapted to drive a piston rod, thereby causing a set dose to be injected by the injection device,
a locking member being operatively connected to the driving element in such a way that movement of the driving element during injection of a set dose causes a movement of the locking member, a position of the locking member thereby being indicative of a remaining amount of liquid in a cartridge of the injection device, the locking member further being adapted to assume a locking position when the remaining amount of liquid is below a predefined critical lower value,
wherein the locking member, when in the locking position, prevents the dose setting member from being operated to set a dose, and wherein the dose setting mechanism may be operated to set and inject a dose when the locking member is not in the locking position, even if injection of the set dose results in the remaining amount of liquid falling below the predefined critical lower value.

2. The dose setting mechanism according to claim 1, wherein the locking member is adapted to move along a longitudinal axis of the injection device during injection of a set dose.

3. The dose setting mechanism according to claim 1, wherein the locking member is further operatively connected to the dose setting member in such a way that movement of the dose setting member during dose setting causes a movement of the locking member.

4. The dose setting mechanism according to claim 3, wherein the locking member is adapted to perform a substantially translational movement along a longitudinal axis of the injection device during injection of a set dose, and adapted to perform a rotational movement about said longitudinal axis during setting of a dose.

5. The dose setting mechanism according to claim 4, wherein the locking member comprises a set of teeth being adapted to engage one or more mating teeth in such a way that rotational movement of the locking member is prevented, the engagement of said teeth thereby defining the locking position of the locking member.

6. The dose setting mechanism according to claim 1, wherein the locking position of the locking member is a position in which the locking member prevents a decoupling of the dose setting member and/or the driving element from a housing of the injection device.

7. An injection device comprising a dose setting mechanism according to claim 1.

8. The dose setting mechanism according to claim 2, wherein the locking member is further operatively connected to the dose setting member in such a way that movement of the dose setting member during dose setting causes a movement of the locking member.

9. The dose setting mechanism according to claim 8, wherein the locking member is adapted to perform a substantially translational movement along a longitudinal axis of the injection device during injection of a set dose, and adapted to perform a rotational movement about said longitudinal axis during setting of a dose.

10. The dose setting mechanism according to claim 9, wherein the locking member comprises a set of teeth being adapted to engage one or more mating teeth in such a way that rotational movement of the locking member is prevented, the engagement of said teeth thereby defining the locking position of the locking member.

11. An injection device comprising a dose setting mechanism according to claim 2.

12. An injection device comprising a dose setting mechanism according to claim 3.

13. An injection device comprising a dose setting mechanism according to claim 4.

14. An injection device comprising a dose setting mechanism according to claim 5.

15. An injection device comprising a dose setting mechanism according to claim 6.

16. An injection device comprising a dose setting mechanism according to claim 8.

17. An injection device comprising a dose setting mechanism according to claim 9.

18. An injection device comprising a dose setting mechanism according to claim 10.
